(19) United States
(12) Patent Application Publication
(10) Pub. No.: US 2010/0268221 A1
(43) Pub. Date: Oct. 21, 2010

(54) SIGNAL DEVICE FOR ELECTROSURGICAL INSTRUMENTS, ADAPTOR FOR CONNECTING AN ELECTROSURGICAL INSTRUMENT

(76) Inventors: Jürgen Beller, Gomaringen (DE); Florian Eisele, Freiburg (DE)

Correspondence Address: DICKSTEIN SHAPIRO LLP, 1825 EYE STREET NW, Washington, DC, 20006-5403, US

(21) Appl. No.: 12/741,659

(22) PCT Filed: Oct. 15, 2008
(86) PCT No.: PCT/EP08/08737

§ 371 Date: Jun. 30, 2010

(30) Foreign Application Priority Data
Nov. 8, 2007 (DE) 10 2007 053 336.7

(51) Int. Cl. A61B 18/12

(52) U.S. Cl. 606/34

(57) ABSTRACT

An electrosurgical instrument comprising an indicator device for displaying at least one piece of status information regarding a control unit. The indicator device is arranged on the electrosurgical instrument and oriented in the distal direction thereof such that, in an active state, the indicator device illuminates an operation area of the electrosurgical instrument. In use, the indicator device fulfils a dual functions. First, the indicator device provides information and second, it simplifies the medical intervention by illuminating the operation area.

### FIELD OF THE INVENTION

Embodiments of the invention relate to an electrosurgical instrument comprising at least one indicator device for displaying at least one piece of status information regarding a control unit and an adaptor for connecting an electrosurgical instrument.

### BACKGROUND

Electrosurgical instruments have been used for many years in high frequency (HF) surgery to coagulate or cut biological tissue. During coagulation, a high frequency current is passed through the tissue to be treated, so that said tissue becomes altered due to protein coagulation and dehydration. In the process, the tissue contracts in such a manner that vessels are closed and bleeding is stemmed. Following coagulation, the tissue can be entirely parted, avoiding severe bleeding, either with the aid of high frequency current or by mechanical means.

Electrosurgical procedures can be carried out with both monopolar and bipolar instruments. In the case of the monopolar technique, the current path typically runs from the electrosurgical instrument to a neutral electrode via the tissue to be treated. Alternatively, bipolar instruments configured with two sections that are electrically isolated from one another can be used. In this case, the current path runs from a first section of the electrosurgical instrument to a second section of the electrosurgical instrument via the tissue to be treated.

A high level of ease of use is important for the successful performance of an operation with an electrosurgical instrument. The electrosurgical instrument should be capable of being operated intuitively and all the necessary information should be easily accessible. This information comprises, in particular, status information provided, for example, by an HF generator.

Particularly problematic is the fact that often times a plurality of electrosurgical instruments is connected to an HF generator. In these cases, a selection must be made as to which instrument is to be supplied with an HF voltage. Assignment of the outputs of the HF generator to a specific instrument, whether a bipolar clamp or a monopolar hook electrode, is frequently difficult.

Various electrosurgical instruments are known from the prior art.

DE 36 27 221 A1 discloses, by way of example, a handle for an electrosurgical instrument that comprises an optical power indicator. This power indicator on the handle provides information about the instrument's power output, for example, the application of a high or low voltage.

DE 40 12 545 A1 discloses a handle for an electrosurgical instrument, characterised in that on the electrode-side end of the handle, a light outlet aperture is provided which is fed by a cold light generator arranged in the HF generator. The light source serves to better illuminate the operation area.

DE 199 43 792 A1 discloses an electrosurgical instrument wherein the handle arranged thereon has an indicator device that enables operation-specific data, for example, the operating mode, to be read from the electrosurgical instrument.

The instruments described above are highly complex to construct.

### SUMMARY

Accordingly, it is an object of the disclosed embodiments to provide an electrosurgical instrument that is easy and comfortable to use.

This goal is achieved with an electrosurgical instrument comprising at least one indicator device for displaying at least one piece of status information of a control unit, wherein the indicator device is arranged on the electrosurgical instrument and oriented in the distal direction thereof such that, in an active state, the indicator device illuminates an operation area of the electrosurgical instrument.

The indicator device can be a single light emitting diode or an array, or matrix, of light emitting diodes. The indicator device preferably comprises active elements, which disclose at least one status of: a control unit and/or HF generator and/or the electrosurgical instrument. In one exemplary embodiment, an active condition of the indicator device can be defined as the condition in which the indicator device is illuminated. Preferably, the active condition of the indicator device correlates with an active condition of the electrosurgical instrument in which said instrument is immediately operational, for example, a current for carrying out a coagulation procedure is being applied.

Thus, an advantage of the disclosed embodiments of the invention is that the indicator is used not only to convey to the user of the device or of the electrosurgical instrument information about the status thereof, but simultaneously to perform an illumination function, which is helpful during the intervention performed with the electrosurgical instrument.

In one exemplary embodiment, the electrosurgical instrument comprises a handle, wherein the at least one indicator device is arranged at the distal end of the handle. This means that the indicator device is arranged to radiate in the direction of the tool head of the electrosurgical instrument and therefore illuminates the region where the electrosurgical instrument is to be used. The described electrosurgical instrument has particular advantages in the case of a hand-held instrument.

In another exemplary embodiment, the indicator device is configured for emitting colored light. The different colors can be used to indicate different pieces of status information or to identify different electrosurgical instruments. For example, the color of the indicator device of the electrosurgical instrument can correspond to a color at the HF generator in order to indicate that, from a plurality of electrosurgical instruments, the present instrument is active or that an HF voltage is being applied to the present electrosurgical instrument.

In another exemplary embodiment, the at least one piece of status information shows the application of a voltage to the electrosurgical instrument, a power value and/or an operating mode of the control unit. As mentioned above, it is useful for the application of a voltage to be indicated if there is a need to distinguish among a plurality of instruments that are partially or completely attached to the same HF generator. Even if only one electrosurgical instrument is used, it can be useful to indicate the active state of the electrosurgical instrument to avoid faulty operation. The color indication can be used to visualise a current strength, a voltage, a frequency and/or a particular frequency pattern. For example, it has been found that, for the coagulation of tissue, a different voltage pattern is required (first operating mode) than the voltage pattern that is used for parting a corresponding tissue (second operating mode). The colored identification could therefore serve to differentiate between operating modes.

In a further exemplary embodiment, the indicator device is coupled to a supply voltage of the electrosurgical instrument. The indicator device can therefore be fed partially, or completely, by the supply voltage applied to the electrosurgical instrument, for example, for a coagulation process.

The problem described above is also solved by an adaptor for connecting an electrosurgical instrument, wherein the adaptor comprises: at least one supply line for providing a supply voltage for the electrosurgical instrument; and an indicator device which is connected to the at least one supply line.

An advantageous distinction between different electrosurgical instruments can also be provided in that the adaptor to which the corresponding electrosurgical instrument is attached comprises an indicator device, which identifies the corresponding instrument or adaptor. The indicator device on the adaptor can also serve to provide status information for the user of the electrosurgical instrument. The adaptor can be, for example, the end of a supply cable of the electrosurgical instrument, wherein the adaptor preferably comprises a plurality of connections for connecting the instrument. In one exemplary embodiment, the adaptor can comprise control elements.

In another exemplary embodiment, the at least one supply line is configured for providing an HF voltage. This means that the adaptor provides the supply voltage for the electrosurgical instrument and is preferably electrically connected to the HF generator.

In another exemplary embodiment, the indicator device is configured to emit colored light. The indicator device may be configured to emit a plurality of different colors, for example, red, green, and blue. The individual colors serve to communicate different status information. Alternatively, a plurality of adaptors can be provided, wherein one adaptor is suitable for emitting a particular color. Differentiation of a plurality of adaptors and thus of the electrosurgical instruments connected thereto can be achieved in this case by means of the color.

Preferably, the indicator device is an active light element that emits light independently. Alternatively, the indicator device can be provided by means of a light waveguide that carries the light waves that are generated to the adaptor. For example, the light wave generator can be provided within the HF generator. When the adaptor is connected to an HF generator, the light source allocated to the connection is automatically coupled in.

In another exemplary embodiment, the indicator device is configured to indicate a voltage applied, in particular an HF voltage. The status information given by the indicator device can therefore show whether the attached electrosurgical instrument, or the adaptor, is active, i.e. is being supplied with a suitable voltage.

### BRIEF DESCRIPTION OF THE DRAWINGS

Embodiments of the invention will now be described in greater detail with reference to the drawings, in which:

FIG. 1 shows a schematic representation of a bipolar electrosurgical instrument;

FIG. 2 shows the relevant components for controlling the electrosurgical instrument of FIG. 1;

FIG. 3 shows the electrical connections between a bipolar electrosurgical instrument and an HF generator;

FIG. 4 shows a circuit diagram of a first indicator device;

FIG. 5 shows a circuit diagram of a second indicator device;

FIG. 6 shows a circuit diagram of a third indicator device; and

FIG. 7 shows the electric cabling between a monopolar electrosurgical instrument and an HF generator.

### DETAILED DESCRIPTION

In the description which follows, the same reference signs are used for the same and similarly acting parts.

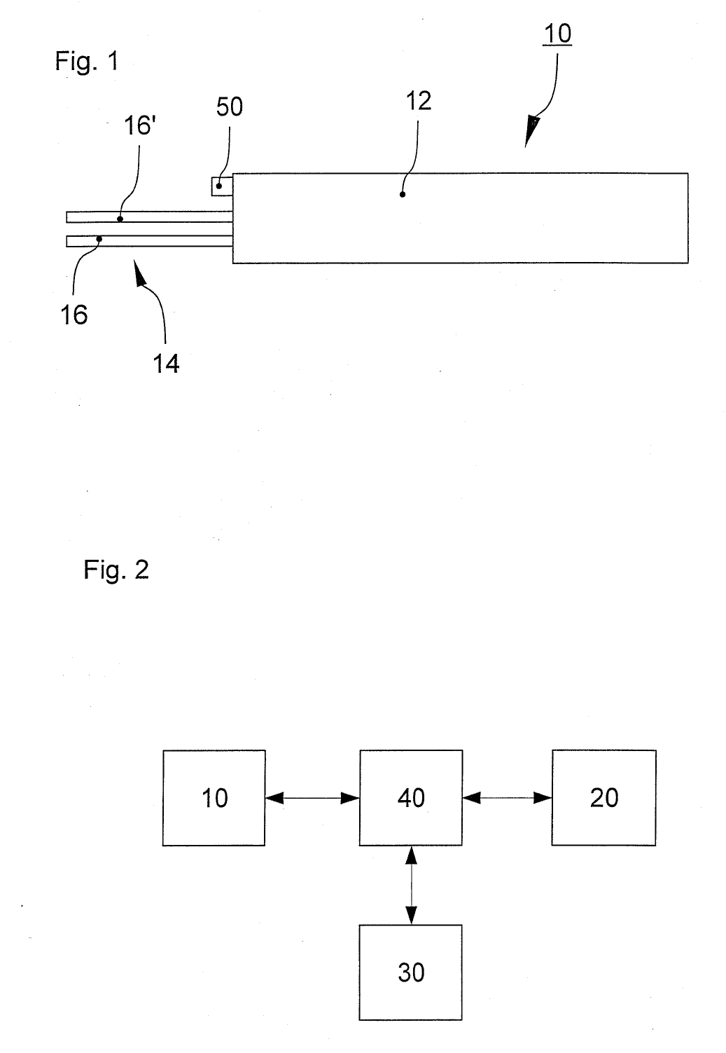

FIG. 1 shows an electrosurgical instrument 10 consisting of a handle 12 and a bipolar instrument head 14. The handle 12 has an elongate form, wherein at the distal end thereof, the bipolar instrument head 14 is attached for performing a surgical procedure. The instrument head 14 has a first electrode 16 and a second electrode 16′ to which a voltage is applied for coagulation of, or for cutting, tissue. The electrosurgical instrument 10 also comprises, at the distal end of the handle 12, an indicator device 50 which is configured to illuminate the bipolar instrument head 14 and a distal operating area. The indicator device 50 is also configured to display one piece of status information. In the present example, the indicator device 50 indicates whether a voltage is applied to the electrodes 16, 16′.

The electrosurgical system according to an embodiment of the invention can be roughly divided into the following four components (see FIG. 2): the electrosurgical instrument 10; an HF generator 20; a foot switch 30; and a control unit 40.

In order to supply the electrosurgical instrument 10 with a suitable voltage and/or a suitable frequency pattern for the respective surgical operation, the system includes the control unit 40, which acts on the HF generator 20 such that the HF generator 20 makes a corresponding output available. To affect the behavior of the control unit 40, the system comprises an input device, particularly foot switch 30. Using the foot switch 30, for example, a particular operating mode can be set for a particular instrument 10. The foot switch 30 can also be used to select one instrument 10, from a plurality thereof connected to the HF generator 20, which is then supplied with a corresponding HF voltage from the generator 20.

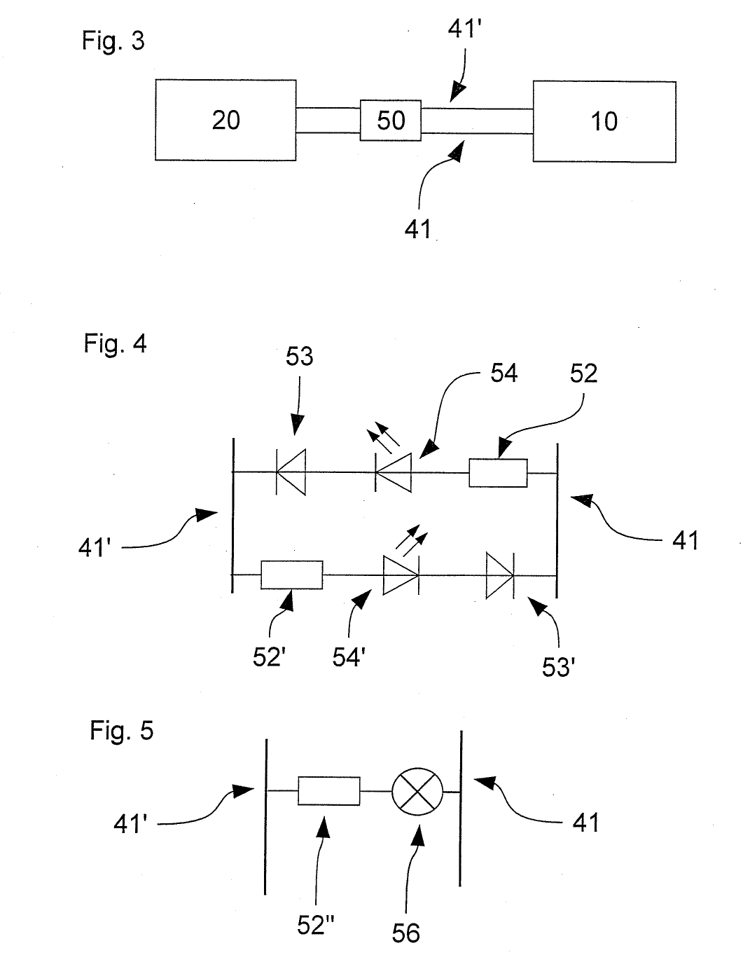

The indicator device 50 provides information regarding the control unit 40, the HF generator 20 or the electrosurgical instrument 10. In particular, according to an embodiment of the invention, the indicator device 50 can display the activation state of the electrosurgical instrument 10. For this purpose, as shown in FIG. 3, the indicator device 50 is coupled to the electrical connection between the HF generator 20 and the electrosurgical instrument 10. FIG. 3 shows a first embodiment wherein the indicator device 50 is arranged on a bipolar electrosurgical instrument 10. In a combination of this type, there are usually two electrical lines running directly between the HF generator 20 and the electrosurgical instrument 10. In the embodiment shown in FIG. 3, these are HF lines 41, 41′.

As shown in FIG. 4, the indicator device 50 can comprise a first light emitting diode 54 and a second light emitting diode 54′, wherein the light emitting diodes 54, 54′ are each arranged parallel to the electrodes 16, 16′ of the bipolar instrument head 14 on the first HF line 41 and the second HF line 41′. The supply lines of the electrosurgical instrument 10 supply the indicator device 50 with current. Protective diodes 53, 53′ are connected on the line side in series with each of the light emitting diodes 54, 54′ to ensure operation of the indicator device 50 even with the typical alternating voltage. Suitably arranged resistors 52, 52′ regulate the voltage that is fed to the indicator device 50, including the two light emitting diodes 54, 54′. A first and second light emitting diode thread are therefore connected in a series circuit, comprising a resistor 52 or 52′, a light emitting diode 54 or 54′ and a protective diode 53 or 53′. The protective diodes 53, 53′ are arranged and configured such that in the first light emitting diode thread, an electric current flows in a first flow direction and in the second thread, an electric current flows in a second flow direction, opposing the first flow direction.

Another embodiment of the indicator device 50 is illustrated in FIG. 5. In this embodiment, an incandescent lamp 56 is coupled between the first and second HF lines 41, 41′ with a corresponding resistor 52″. Rectification of the supply current is not necessary.

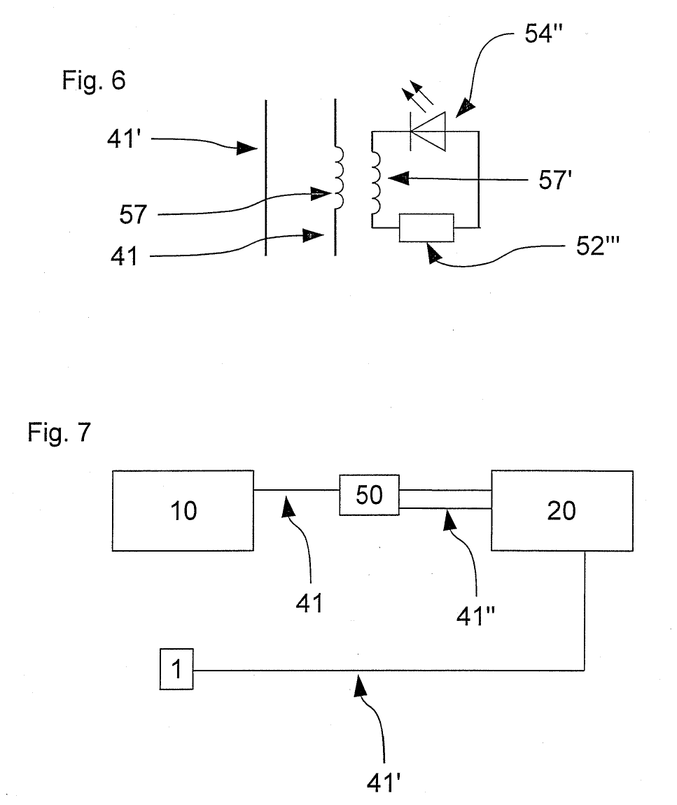

FIG. 6 shows the inductive coupling of the indicator device 50 into the supply current circuit of the bipolar instrument head 14. For this purpose, the first HF line 41 has a first induction coil 57, which is magnetically coupled to a second induction coil 57′. The energy induced in the second induction coil 57′ is used to operate a second light emitting diode 54″, which has a resistor 52′ connected on the line side thereof.

Alternatively, the indicator device 50 can be used with monopolar electrosurgical instruments 10. FIG. 7 shows a suitable arrangement. Normally, the monopolar instrument is supplied directly with the voltage from the HF generator 20 via a first HF line 41. A second HF line 41′ contacts a neutral electrode 1, which is directly in contact with the patient. To operate the indicator device 50 according to FIG. 4 or 5, a further line 41″, which supplies the indicator device 50 must be provided. In this embodiment, the indicator device 50 is fed with the supply voltage for the electrosurgical instrument. For the inductive coupling of the indicator device 50, an HF line 41″ can be dispensed with.

The indicator device 50 described above can be integrated, according to an embodiment of the invention, into an adaptor to which an electrosurgical instrument 10 can be connected. Preferably, the handle 12 of the electrosurgical instrument 10 can be plugged into corresponding connections of the adaptor. In this embodiment, the indicator device also uses the supply voltage of the electrosurgical instrument 10 to operate the associated light emitting diodes 54, 54′, 54″ or a corresponding incandescent lamp 56. The circuits shown in FIGS. 4-6 can be adapted accordingly for use in the adaptor.

In another embodiment, the indicator device 50 of the adaptor is supplied with chromatic light waves via a light waveguide running alongside the supply lines for the instrument 10. The HF generator 12 can be adapted such that if a voltage exists on the adaptor and therefore also on the plugged-in electrosurgical instrument 10, the indicator device 50 is fed with suitably colored light. Alternatively, the HF generator 20 and the control unit can be configured such that light waves are provided for a particular adaptor among a plurality of adaptors connected to the HF generator 20 only when a current flows at the relevant supply voltage. A pre-determined light color at the indicator device 50 can correspond to a light color at the HF generator 20, in order to indicate that a voltage is being applied to precisely this adaptor.

In a further embodiment, the light emitting diodes 54, 54′, 54″ can be adapted to emit colored light that corresponds, for example, to an indicator color of an indicator at the HF generator 20.

1. An electrosurgical instrument comprising:
at least one indicator device for displaying at least one piece of status information regarding a control unit,
wherein the indicator device is arranged on the electrosurgical instrument and oriented in a distal direction thereof such that, in an active state, the indicator device illuminates an operation area of the electrosurgical instrument, and
wherein the indicator device is coupled to a supply voltage of the electrosurgical device such that the supply voltage is fed to the indicator device.

2. The electrosurgical instrument according to claim 1, further comprising a handle,
wherein the at least one indicator device is arranged at a distal end of the handle.

3. The electrosurgical instrument according to claim 1, wherein the indicator device is configured for emitting colored light.

4. The electrosurgical instrument according to claim 1, wherein the at least one piece of status information shows the application of a voltage to the electrosurgical instrument, a power value and/or an operating mode of the control unit.

5. (canceled)

6. A plurality of adaptors, each for connecting to an electrosurgical instrument (10), in particular an electrosurgical instrument according to claim 1, each adaptor comprising:
at least one supply line for providing a supply voltage for the electrosurgical instrument; and
an indicator device, which is connected to the at least one supply line,
wherein an adaptor for differentiating each of the electrosurgical instruments is configured for emitting a particular colored light.

7. The adaptors according to claim 6, wherein the at least one supply line is configured for providing a high frequency voltage.

8. (canceled)

9. The adaptors according to claim 6, further comprising a handle.

10. The adaptors according to claim 6, wherein the indicator device is configured to display a voltage applied to the device.

11. The adaptors according to claim 10, wherein the voltage applied is a high frequency voltage.
